(19) United States
(12) Patent Application Publication
(10) Pub. No.: US 2026/0014305 A1
(43) Pub. Date: Jan. 15, 2026

(54) EXTRACORPOREAL CIRCULATION DEVICE, INCLUDING MANAGEMENT DEVICE, AND METHOD OF OPERATION

(71) Applicant: TERUMO KABUSHIKI KAISHA, Tokyo (JP)

(72) Inventors: Nobutaka Shimizu, Kanagawa (JP); Shuhei Tomita, Shizuoka (JP)

(21) Appl. No.: 19/336,598

(22) Filed: Sep. 23, 2025

Related U.S. Application Data
(63) Continuation of application No. PCT/JP2024/008829, filed on Mar. 7, 2024.

(30) Foreign Application Priority Data
Mar. 24, 2023 (JP) 2023-0048918

(51) Int. Cl. A61M 1/36

(52) U.S. Cl. CPC A61M 1/3601; A61M 1/3666; A61M 2202/0021; A61M 2202/0413; A61M 2205/18; A61M 2205/502; A61M 2205/584

(57) ABSTRACT

A device including a display unit that is easily visually recognized by a user, an extracorporeal circulation device including the management device. A management device manages an extracorporeal circulation device including an oxygenation device, a pump unit, and an extracorporeal circulation circuit IR, and includes a display unit that displays various types of information regarding the extracorporeal circulation device, the display unit includes a first display area in which fixed information that is fixed in advance is displayed, and a second display area configured in such a manner that the displayed information is changeable, in which trend graph information of the fixed information displayed in the first display area is displayed in the second display area, and non-fixed information related to the fixed information is displayed.

### CROSS REFERENCE TO RELATED APPLICATIONS

This application is a continuation of PCT Application No. PCT/JP2024/008829, filed Mar. 7, 2024, based on and claiming priority to Japanese Application No. JP2023-0048918, filed Mar. 24, 2023, both of which are incorporated herein by reference in their entirety.

### BACKGROUND OF THE INVENTION

The present invention relates to, for example, a management device that manages pulmonary circulation and the like for oxygenating blood of a patient outside the body and returning the oxygenated blood into the body and a method of operation.

Conventionally, for example, an extracorporeal circulation device that performs pulmonary circulation or the like in which blood of a patient is drawn out of the body, oxygenated by an oxygenator or the like, and returned into the body again has been used.

Such an extracorporeal circulation device includes a management device that manages the extracorporeal circulation device, and the management device includes a display (graphical user interface or the like) that displays various data related to the extracorporeal circulation device.

On the other hand, in recent years, with the demand for downsizing of the extracorporeal circulation device, the display of the management device has also been downsized.

However, even if the display is downsized, the number of pieces of essential data and the like that must be displayed at the same time is not reduced, and thus the essential number of pieces of data is simultaneously displayed on a smaller display. In this case, the data is displayed in an overlapping manner, and therefore, there is a problem that it is difficult for a doctor who uses an extracorporeal circulation device or the like, a medical engineer (ME), or the like to visually recognize the data displayed on the display.

### SUMMARY OF THE INVENTION

Therefore, an object of the present invention is to provide a management device including a display unit such as a display that is easily visually recognized by a user, and an operation method of the extracorporeal circulation device.

An aspect of the present invention is a management device that manages an extracorporeal circulation device including an oxygenation device, a pump unit, and an extracorporeal circulation circuit, in which the management device includes a display unit that displays various types of information regarding the extracorporeal circulation device, the display unit includes a first display area in which fixed information that is information fixed in advance is displayed, and a second display area configured in such a manner that displayed information is changeable, and trend graph information generated from the fixed information displayed in the first display area is displayable in the second display area, and non-fixed information related to the fixed information is displayable.

With the above configuration, numerical information of fixed information such as a flow rate, a rotation speed of a pump, and FI, which are important information of the extracorporeal circulation device and the like are always displayed in the first display area of the display unit, and non-fixed information such as a trend graph or the like of the fixed information within a predetermined time or a value of a pressure gauge of the extracorporeal circulation circuit is not always displayed in the second display area, and the display can be changed as necessary.

Therefore, the user can quickly and reliably grasp necessary information even with a small display.

In particular, when a low-skilled person operates an extracorporeal circulation device requiring skill, there is a case where a numerical value or the like to be visually recognized first cannot be easily recognized.

On the other hand, this demand is particularly high at a site where the extracorporeal circulation device is used with high urgency.

In this regard, in the above configuration, even a low-skilled person can easily and quickly recognize a numerical value or the like to be visually recognized first.

In the above management device an aspect of the invention includes a setting screen of the non-fixed information displayed in the second display area is displayable instead of the non-fixed information.

With the above configuration, since the setting screen of the non-fixed information displayed in the second display area is displayable instead of the non-fixed information, the user can easily and quickly perform setting and the like.

In the above management device an aspect of the invention includes the setting screen has a time display area, and in the time display area, elapsed time information starting from a current setting time is displayable.

With the above configuration, it is possible to instantaneously grasp the elapsed time from the previous set time without calculating the elapsed time.

In the above management devices, a time axis of time information for displaying the trend graph information is changeable.

With the above configuration, since the time axis of the time information for displaying the trend graph information is changeable, the user can display the trend graph information by changing the time axis to a necessary time axis (for example, 24 hours, 12 hours, and the like).

The above management device may include an abnormal value information detection unit that detects abnormal value information regarding the fixed information and the non-fixed information, in which the fixed information and the non-fixed information are displayed by being divided by different frame parts for each of the fixed information and the non-fixed information, and when the abnormal value information detection unit detects an abnormality in at least one of the fixed information or the non-fixed information, the abnormal value information detection unit displays an abnormal frame part, which is the frame part for at least one of the fixed information or the non-fixed information in which the abnormality is detected, in an alarm color different from a background color of the display unit.

With the above configuration, the abnormal frame part, which is a frame part for at least one of the fixed information in which an abnormality is detected by the abnormal value information detection unit or the non-fixed information, is set to the alarm color, which is a color different from the background color of the display unit, so that the user can quickly grasp information of the abnormal value.

In the above management device, the alarm color is displayed in a plurality of different colors depending on a degree of priority of the alarm.

With the above configuration, since the alarm color has a plurality of different colors (red, yellow, and the like) depending on the degree of priority of the alarm, the user can instantaneously determine the degree (priority) of the alarm with the color.

In the above management device, an icon part indicating abnormality content may be displayed in the abnormal frame part.

With the above configuration, since the icon part indicating abnormality content is displayed in the abnormal frame part, the user can instantaneously grasp the content of the abnormal value by visually recognizing the icon part.

In the above management device, the icon part may be an upward arrow when the abnormality content exceeds an upper limit value, and is a downward arrow when the abnormality content exceeds a lower limit value.

With the above configuration, the icon part is an upward arrow when the abnormal value exceeds the upper limit value, and is displayed as a downward arrow when the abnormal value exceeds the lower limit value, so that the user can instantaneously grasp the content of the abnormality.

The management device may include an abnormality information display part that displays abnormality information content related to the abnormal frame part, in which the abnormality information display part is configured to be scrolled and displayed, and the abnormality information display part is configured to simultaneously display information of a number of pieces of abnormal value information detected by the abnormal value information detection unit within a certain period of time.

With the above configuration, since the abnormality information display part (bubble detection alarm or the like) that displays abnormality information content related to the abnormal frame part is provided, the user can more specifically grasp the abnormality content and the number thereof.

Further, with the above configuration, since the number information of all the pieces of abnormal value information detected by the abnormal value information detection unit is also displayed at the same time, the number of pieces of abnormal value information can also be accurately grasped.

In the management device above, when a plurality of pieces of pressure information is displayed as the non-fixed information in the second display area, a plurality of pieces of the pressure information is displayed in parallel in an order corresponding to a blood sending direction of the extracorporeal circulation device.

With the above configuration, when the plurality of pieces of pressure information is displayed as the non-fixed information in the second display area, the plurality of pieces of pressure information is displayed in parallel in the order corresponding to the blood sending direction of the extracorporeal circulation device. In this way, the user can easily grasp in which part of the extracorporeal circulation device the pieces of pressure information are located.

In the above management device, a plurality of types of the non-fixed information may be switched to the second display area and displayed in a predetermined order.

With the above configuration, since the plurality of types of non-fixed information (numerical screen, trend graph, and the like) is switched to the second display area and displayed in the predetermined order, the user can easily and quickly grasp all the plurality of pieces of non-fixed information with a size that is easy to visually recognize.

An object is achieved, in the present invention, by an extracorporeal circulation device, the extracorporeal circulation device including an oxygenation device, a pump unit, an extracorporeal circulation circuit, and a management device, in which the management device includes a display unit that displays various types of information regarding the extracorporeal circulation device, the display unit includes a first display area in which fixed information that is information fixed in advance is displayed, and a second display area configured in such a manner that displayed information is changeable, and trend graph information generated from the fixed information displayed in the first display area that is displayable in the second display area, and non-fixed information related to the fixed information that is displayable.

An object is achieved, in the present invention, by an operation method of a management device that manages an extracorporeal circulation device including an oxygenation device, a pump unit, and an extracorporeal circulation circuit, in which the management device includes a display unit that displays various types of information regarding the extracorporeal circulation device, the display unit includes a first display area in which fixed information that is information fixed in advance is displayed, and a second display area configured in such a manner that displayed information is changeable, and trend graph information generated from the fixed information displayed in the first display area that is displayable in the second display area, and non-fixed information related to the fixed information that is displayable.

As described above, according to the present invention, there is an advantage that it is possible to provide a management device including a display unit such as a display that is easily visually recognized by a user, and an operation method of the extracorporeal circulation device.

### BRIEF DESCRIPTION OF THE DRAWINGS

FIG. 1 is a schematic diagram illustrating a configuration of an extracorporeal circulation device according to an embodiment of the present invention.

FIG. 2 is a schematic block diagram illustrating a configuration of a controller and sensors.

FIG. 3 is a schematic explanatory diagram illustrating a first screen.

FIG. 4 is a schematic explanatory diagram illustrating a second screen.

FIG. 5 is a schematic diagram illustrating a screen example of a lower portion of a lower display of a third screen.

FIG. 6 is a schematic explanatory diagram illustrating a fourth screen displaying information of an optional flow rate sensor on a display.

FIG. 7 is a schematic explanatory diagram illustrating the fourth screen for displaying information of the optional flow rate sensor on the display.

FIG. 8 is a schematic explanatory diagram illustrating a screen example in which a time axis is changed to 24 hours.

FIG. 9 is a schematic explanatory diagram illustrating a screen example in which the time axis is changed to 12 hours.

FIG. 10 is a schematic explanatory diagram illustrating a screen example of a pressure setting screen.

FIG. 11 is a schematic explanatory diagram illustrating a pressure alarm setting screen.

FIG. 12 is a schematic explanatory diagram illustrating a screen example of an oxygen saturation alarm setting screen.

FIG. 13 is a schematic explanatory diagram illustrating an SO2-1 alarm setting screen.

FIG. 14 is a schematic explanatory diagram illustrating a screen example of a hemoglobin/hematocrit value setting screen.

FIG. 15 is a schematic diagram illustrating a hematocrit value setting screen.

FIG. 16 is a schematic explanatory diagram illustrating a mode of elapsed time display of CAL implementation.

FIG. 17 is a schematic explanatory diagram illustrating a display in a state in which an alarm is displayed.

FIGS. 18A and 18B are schematic explanatory diagrams illustrating examples in which three pieces of abnormality content text information are displayed on the display.

FIGS. 19A, 19B and 19C are schematic explanatory diagrams illustrating examples in which four pieces of abnormality content text information are displayed on the display.

### DETAILED DESCRIPTION OF EMBODIMENTS

Hereinafter, preferred embodiments of the present invention will be described in detail with reference to the accompanying drawings.

Note that the embodiment described below is a preferred specific example of the invention, and thus various technically preferable limitations are given. However, the scope of the invention is not limited to these aspects unless there is a statement to limit the invention in the following description.

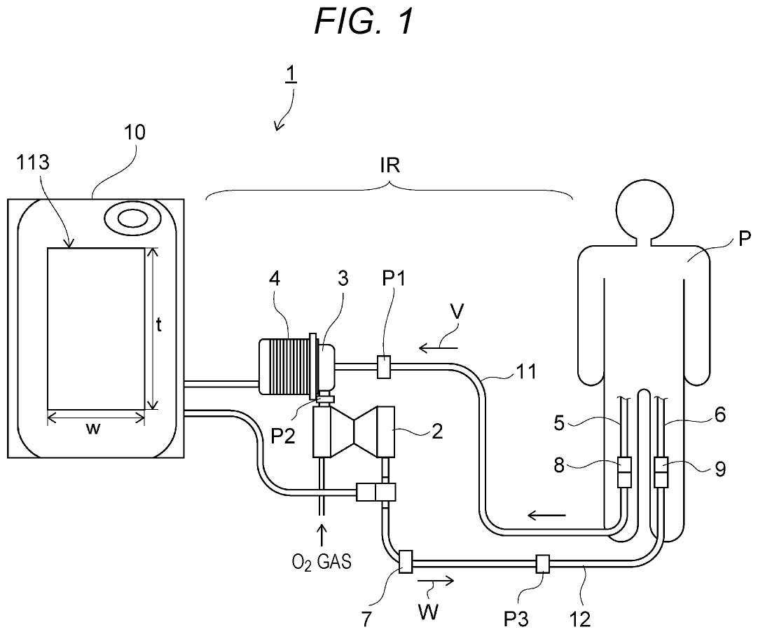

FIG. 1 is a schematic diagram illustrating a configuration of an extracorporeal circulation device 1 according to an embodiment of the present invention.

As illustrated in FIG. 1, the extracorporeal circulation device 1 is a device that performs extracorporeal circulation of blood of a subject, for example, a patient P.

The patient P when using the extracorporeal circulation device 1 may be in a situation where the heart does not operate normally, or in a situation where the heart operates normally but the lungs do not operate normally.

The extracorporeal circulation device 1 illustrated in FIG. 1 according to the present embodiment may be used, for example, in a situation of where a cardiac surgery of the patient P is performed or in a subsequent treatment in an ICU (intensive care unit).

Specifically, a centrifugal pump 3 is operated via a drive motor 4 of the extracorporeal circulation device 1 to perform oxygenator extracorporeal blood circulation in which blood is removed from a vein (vena cava) of the patient P, and after the blood is oxygenated by performing gas exchange in the blood using, for example, an oxygenator 2, which is an oxygenation device, the blood is returned to an artery (aorta) of the patient P again. That is, the extracorporeal circulation device 1 is a device that substitutes for the heart and the lungs.

Further, the example of the extracorporeal circulation device 1, illustrated in FIG. 1, has the following configuration. The extracorporeal circulation device 1 includes a circulation circuit IR, for example, which is an extracorporeal circulation circuit that circulates blood, and the circulation circuit IR includes an oxygenator 2, a centrifugal pump 3, for example, which is a pump unit, a drive motor 4, a venous side catheter (blood removal side catheter) 5, an arterial side catheter (blood sending side catheter) 6, and a controller 10, for example, which is a management device.

Note that the centrifugal pump 3 is also referred to as a blood pump, and a pump other than a centrifugal pump can also be used.

The venous side catheter 5 is inserted from the femoral vein via a connector 8, and the distal end of the venous side catheter 5 is placed in the right atrium.

The arterial side catheter 6 is inserted from the femoral artery via a connector 9.

The venous side catheter 5 is connected to the centrifugal pump 3 via the connector 8 by using a blood removal tube 11. The blood removal tube 11 is a channel for sending blood.

When the drive motor 4 operates the centrifugal pump 3 according to a command of the controller 10, the centrifugal pump 3 is configured to return the blood removed from the blood removal tube 11 and passed through the oxygenator 2 to the patient P via a blood feeding tube 12.

The oxygenator 2 is disposed between the centrifugal pump 3 and the blood feeding tube 12. The oxygenator 2 introduces oxygen gas and performs a gas exchange operation (oxygen addition and/or carbon dioxide removal) on the blood.

The oxygenator 2 is, for example, a membrane oxygenator, and may be a hollow fiber membrane oxygenator. The blood feeding tube 12 is a channel that connects the oxygenator 2 and the arterial side catheter 6.

The blood removal tube 11 and the blood feeding tube 12 are, for example, conduits made of synthetic resin having high transparency and flexibility such as vinyl chloride resin and silicone rubber.

In the blood removal tube 11, blood flows in a direction of arrow V, and in the blood feeding tube 12, blood flows in a direction of arrow W.

Further, in the extracorporeal circulation device 1, a flow rate sensor 7 that measures a flow rate value of blood in the blood feeding tube 12, for example, which is extracorporeal circulation information of the patient P, is disposed in the blood feeding tube 12.

In addition, a first pressure sensor P1 (blood removal pressure) for measuring a pressure value that is extracorporeal circulation information is disposed in the blood removal tube 11, a second pressure sensor P2 (oxygenator pressure) is disposed between the centrifugal pump 3 and the oxygenator 2, and a third pressure sensor P3 (blood sending pressure) is disposed in the blood feeding tube 12.

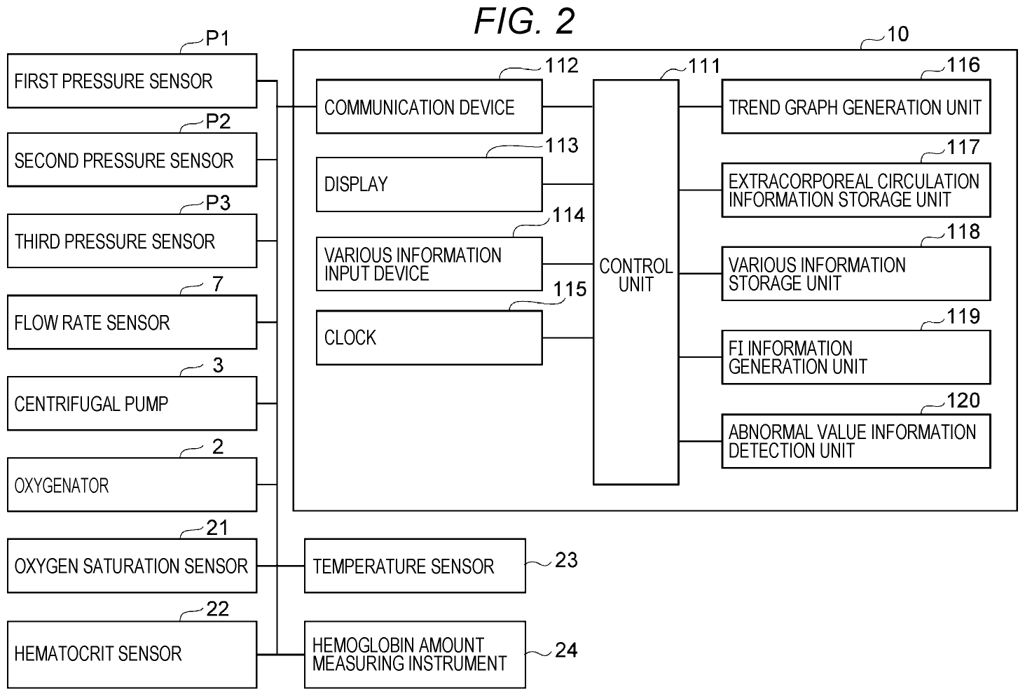

As illustrated in FIG. 2, the controller 10 is connected to an oxygen saturation sensor 21, a hematocrit sensor 22, a temperature sensor 23, and a hemoglobin amount measuring instrument 24 that measure, respectively, oxygen saturation, a hematocrit value, a temperature, and a hemoglobin value, which are extracorporeal circulation information of the patient P.

As illustrated in FIG. 1, the controller 10 includes, for example, a display 113, which is a small display unit for performing color display by color liquid crystal, organic EL (organic light-emitting-diode), or the like, and has a size of, for example, an overall width of 178 mm, a screen width (w) of 91 mm, and a screen height (t) of 152 mm, which is smaller than a conventional screen width (w) of 114 mm and a conventional screen height (t) of 152 mm.

Meanwhile, the controller 10 and the like of the extracorporeal circulation device 1 illustrated in FIG. 1 may include various combinations of electronic hardware and software, for example a computer, with the computer including a central processing unit (CPU), a random access memory (RAM), a read only memory (ROM), and the like (not illustrated), which are connected via a bus (not illustrated).

FIG. 2 is a schematic block diagram illustrating an example of a configuration of the controller 10 and the like of FIG. 1.

The controller 10 includes a control unit 111, with the control unit 111 controlling a communication device 112 for the controller 10 to communicate, the above-described display 113, a various information input device 114, a clock 115 for generating time information, and the like.

As further illustrated in the example of FIG. 2, the control unit 111 also controls a trend graph generation unit 116, an extracorporeal circulation information storage unit 117, a various information storage unit 118, an FI information generation unit 119, and an abnormal value information detection unit 120. Specific contents thereof will be described later.

Examples of information stored in the extracorporeal circulation information storage unit 117, will be described with reference to FIGS. 1 and 2. Information on pressure values of the first pressure sensor P1, the second pressure sensor P2, and the third pressure sensor P3 is stored in the extracorporeal circulation information storage unit 117 in association with the time information of the clock 115. A flow rate value of the flow rate sensor 7 is stored in the extracorporeal circulation information storage unit 117 in association with the time information of the clock 115. The value of the rotation speed of the centrifugal pump 3 is stored in the extracorporeal circulation information storage unit 117 in association with time information of the clock 115.

In addition, the value of the oxygen saturation percentage (%) of the oxygen saturation sensor 21 is also stored in the extracorporeal circulation information storage unit 117 in association with the time information of the clock 115. The value degrees (°) of the body temperature of the temperature sensor 23 is stored in the extracorporeal circulation information storage unit 117 in association with the time information of the clock 115. The value of the hematocrit sensor 22 (volume ratio (%) of red blood cells in the whole blood) is stored in the extracorporeal circulation information storage unit 117 in association with the time information of the clock 115.

In addition, the value of the hemoglobin amount measuring instrument 24 (the amount (g/dL) of hemoglobin in the whole blood) is stored in the extracorporeal circulation information storage unit 117 in association with the time information of the clock 115.

Note that, regarding an FI value, the FI information generation unit 119 operates to correct and convert a circulation amount of blood by a patient body type difference (per body surface area), generate the FI value (L/min/m2), and store the FI value (L/min/m2) in the extracorporeal circulation information storage unit 117 in association with the time information of the clock 115.

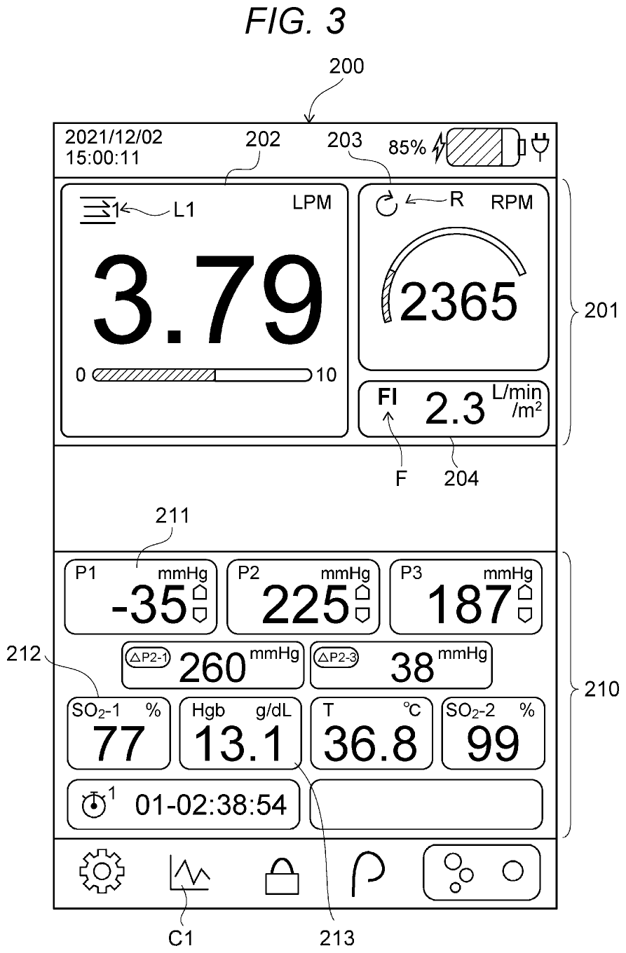

Examples of extracorporeal circulation information displayed on the display 113 will be described with reference to FIGS. 1 and 3. Information is displayed on the display (screen) 113, with FIG. 3 illustrating a schematic example of an explanatory diagram of a first screen 200. The first screen example 200 includes an upper display 201 that defines a first display area and a lower display 210 that defines a second display area.

In the upper display 201, for example, three information display frames are arranged, including a large frame, a medium frame, and a small frame. On the upper display 201, for example, flow rate value, rotation speed of centrifugal pump, and FI value, which are particularly important fixed information for the extracorporeal circulation device 1, are displayed.

The large frame is, for example, a flow rate frame 202 in which numerical information of a flow rate (blood flow rate (L/min)/body surface area (m2)) is displayed. The illustrated value, for example, “3.79” (an example of the numerical information) of the flow rate value is displayed in the flow rate frame 202 with reference to the value of the flow rate sensor 7 of the extracorporeal circulation information storage unit 117 (shown in FIG. 2). Since the flow rate is an important index for grasping the circulation amount of blood, it is displayed in the large frame 202. Specifically, during operation, a medical worker or the like confirms that the flow rate is a constant value as an index of circulation, and when the flow rate is a low numerical value (below a predetermined threshold), it means there is insufficient circulation of blood.

Further, as illustrated in FIG. 3 with reference to FIGS. 1 and 2, characters of “LPM” indicating the flow rate and a flow rate icon L1 are simultaneously displayed together with the numerical information in the flow rate frame 202.

The middle frame is, for example, a centrifugal pump frame 203 in which numerical information of the rotation speed of the centrifugal pump 3 (rotation speed of the centrifugal pump per minute) is displayed. The rotation speed of the centrifugal pump 3 refers to the value (for example, in revolutions per minute (RPMs)) of the centrifugal pump 3 stored in the extracorporeal circulation information storage unit 117, and in the case of FIG. 3, the example of a current value “2365” is displayed. The rotation speed of the centrifugal pump 3 is related to the blood flow rate, and is displayed because it is an important index to be viewed together with the flow rate at a time when there may be concern.

Further, as illustrated in FIG. 3, characters “RPM” indicating the rotation speed of the centrifugal pump 3 and a centrifugal pump icon R are simultaneously displayed together with the numerical information on the centrifugal pump frame 203.

The small frame is, for example, an FI frame 204 in which numerical information of flow index (FI: L/min/m2) is displayed. The FI value (L/min/m2) refers to the FI value of the extracorporeal circulation information in storage unit 117, with an example of a current value “2.3” displayed.

FI is similar to a cardiac index (CI), but CI indicates a circulation state in the body of the patient, whereas FI is different in that the blood volume in the extracorporeal circulation device 1 is obtained. Specifically, FI is an index for grasping hemodynamics, and is an index obtained by correcting and converting the circulation amount of blood by the patient body type difference (per body surface area). That is, FI is one of indices for confirming an appropriate state of the circulating amount of blood. In the FI frame 204, as illustrated in FIG. 3, an FI icon F is simultaneously displayed together with the numerical information of FI, for example “2.3.”

Examples of lower displays 210 and 310 will be described with reference to FIG. 3 in view of FIGS. 1 and 2. In this example, a plurality of two types of screens are illustrated in the lower display 210, and the screens can be switched by the user operating a screen change icon C1.

Examples of the lower display 210 of the first screen 200 are illustrated in FIG. 3, with reference to FIGS. 1 and 2. The lower display 210 of the first screen 200 show examples of current numerical information such as pressure values of the first pressure sensor P1 (blood removal pressure), the second pressure sensor P2 (oxygenator pressure), and the third pressure sensor P3 (blood sending pressure), an oxygen saturation value of the oxygen saturation sensor 21, a temperature value of the temperature sensor 23, a value of the hematocrit sensor 22, and a value of the hemoglobin amount measuring instrument 24, which are non-fixed information related to the fixed information such as the flow rate value, the rotation speed of the centrifugal pump, and the FI value displayed on the upper display 201.

In the screen example of the lower display 210, all the extracorporeal circulation information is displayed with current numerical values, and the screen is effective in a case where the user wants to grasp the current extracorporeal circulation information of the patient as numerical information. Further, the first pressure sensor P1 (blood removal pressure), the second pressure sensor P2 (oxygenator pressure), and the third pressure sensor P3 (blood sending pressure), are displayed on the screen in the order of the actual blood flowing direction (blood removal pressure (P1), oxygenator pressure (P2), and blood sending pressure (P3)) of the extracorporeal circulation from left to right in FIG. 3. Therefore, the user can easily grasp where along the blood flow path the pressure information pertains to.

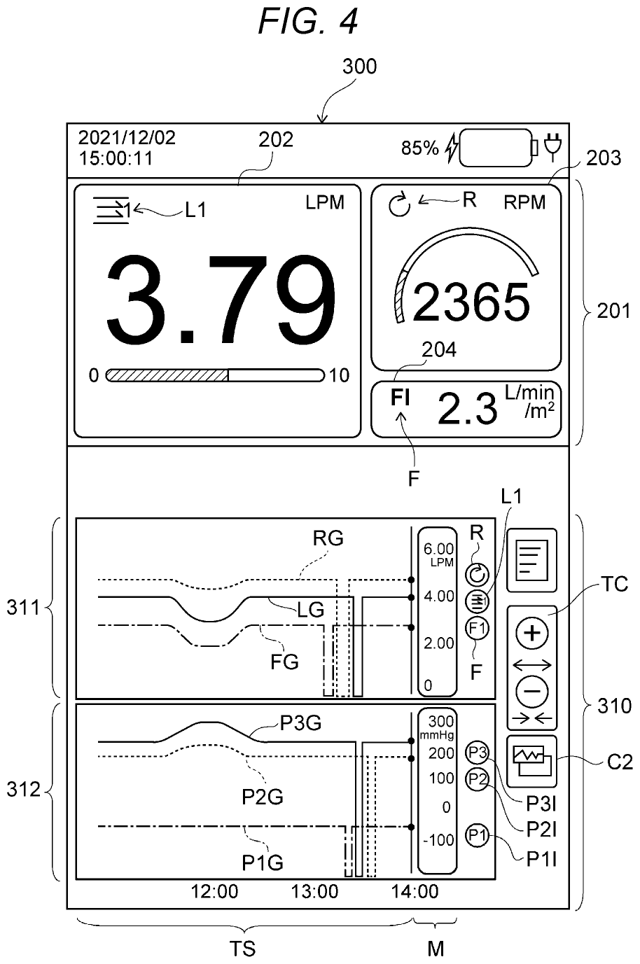

Examples of the lower display 310 of second screen) are illustrated in FIG. 4, with reference to FIGS. 1 and 2, illustrating a second screen 300. The upper display 201 of the second screen 300 is similar to the first screen 200 of FIG. 3, and thus is denoted by the same reference numeral, and description thereof is omitted. Trend graph information of the flow rate value or the like displayed on the upper display 201 is displayed on the lower display 310, and the trend graph information of the first pressure sensor P1 or the like related to the flow rate value or the like, which is fixed information, is also displayed.

Specifically, in this configuration, the lower display 310 further includes an upper-side lower display 311 and a lower-side lower display 312, and trend graph information of the extracorporeal circulation information is displayed on each of the displays 311, 312. The trend graph information of the extracorporeal circulation information is a graph indicating transition of the extracorporeal circulation information within a predetermined time, for example, a flow rate value of a flow rate sensor 7, a rotation speed of a centrifugal pump 3, an FI value, or the like within the predetermined time.

Specifically, a trend graph generation unit 116 in FIG. 2 operates to generate a trend graph such as a flow rate value on the basis of information such as the flow rate value of the extracorporeal circulation information storage unit 117 and displays the trend graph on the lower display 310.

The extracorporeal circulation information generated as the trend graph includes, in addition to the flow rate value and the like, the pressure values of the first pressure sensor P1, the second pressure sensor P2, and the third pressure sensor P3, the oxygen saturation value of the oxygen saturation sensor 21, the temperature value of the temperature sensor 23, the value of the hematocrit sensor 22, the value of the hemoglobin amount measuring instrument 24, and the like.

The main reason for displaying such a trend graph on the display 113 is as follows. In a case where the extracorporeal circulation device 1 is used, the operator does not constantly monitor the patient but periodically monitors the patient in a state where the patient is stable depending on the condition and operation content of the patient. Therefore, a trend graph is displayed in order to grasp a time period in which the operator (user) cannot monitor. Further, when an alarm or the like occurs, the progress before occurrence of an alarm or the like can be confirmed by visually observing the trend graph.

Examples of the upper-side lower display 311 of lower display 310 of second screen 300 are illustrated in FIG. 4, with reference to FIGS. 1 and 2. The upper-side lower display 311 of the lower display 310 of the second screen 300, the flow rate value of the flow rate sensor 7, the rotation speed of the centrifugal pump 3, the FI value, and the like displayed on the upper display 201 in the trend graph are displayed as illustrated in FIG. 4. In this manner, the upper-side lower display 311 displays the trend graph for the same values as the flow rate value, the value of the rotation speed of the centrifugal pump 3, and the FI value displayed on the upper display 201.

Specifically, a flow rate trend graph LG, a rotation speed trend graph RG of the centrifugal pump 3, and a FI value trend graph FG are illustrated.

In particular, the upper display 201 displays the current numerical values such as the flow rate value, and the upper-side lower display 311 displays the trend graph LG and the like in a predetermined time period for values corresponding to the upper display 201, whereby the flow rate value and the like can be grasped more accurately.

Further, on the current value side (the right end in the drawing) of the trend graph of the flow rate value, the value of the rotation speed of the centrifugal pump 3, and the FI value displayed on the upper-side lower display 311, the corresponding flow rate icon L1, centrifugal pump icon R, and FI icon F are arranged close to each other in parallel. The flow rate icon L1 and the like are similar to the flow rate icon L1 and the like displayed on the upper display 201 (see FIG. 3). Therefore, a user (a doctor, a medical engineer (ME)) or the like who views the trend graph can instantly grasp the type of the trend graph by visually recognizing the icon displayed in proximity.

Further, in this configuration, the flow rate icon L1 and the like are arranged without overlapping each other, and even when the displayed trend graph LG and the like intersect in the middle, the flow rate icon L1 and the like are arranged according to the position corresponding to the current value of the trend graph LG, so as to be easily recognized by the user. That is, the user can instantaneously determine which trend graph LG or the like each trend graph LG or the like is by viewing the current value of the trend graph LG or the like.

Further, on the current value side of the trend graph LG or the like, a graph scale M of the trend graph LG or the like is also displayed in parallel and in close proximity. Thus, the user can instantaneously grasp the numerical value indicated by the trend graph LG or the like.

The present embodiment has a configuration in which numerical information of the current values of the flow rate value, the value of the rotation speed of the centrifugal pump, and the FI value, which are the most important information for the extracorporeal circulation device 1, is displayed on the upper display 201, and the trend graph LG and the like such as the same flow rate value is displayed on the upper-side lower display 321 of the lower display 320, so that the user can reliably confirm the most important information.

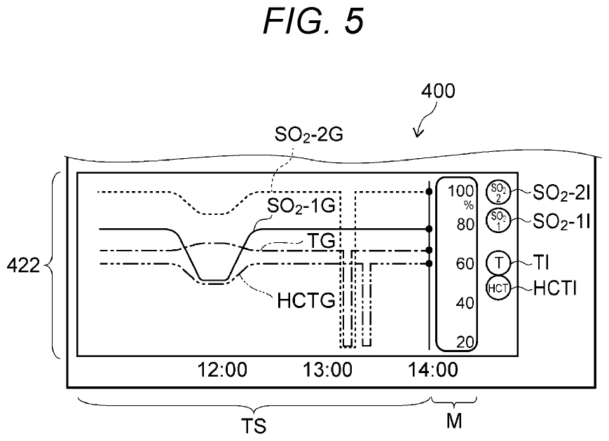

An example of lower-side lower display 312 of lower display 310 of second screen 300 is illustrated in FIG. 4 with reference to FIGS. 1 and 2. The screen of the lower-side lower display 312 of the lower display 310 can be changed to a screen of another trend graph (a screen, for example, of FIG. 5 to be described later) by selecting a screen change icon C2 and changing the screen. These screens will be described below.

An example of a lower-side lower display 312 of second screen 300 is illustrated in FIG. 4 with reference to FIGS. 1 and 2. Trend graphs based on the values of first pressure sensor P1, second pressure sensor P2, and third pressure sensor P3 are displayed on the lower-side lower display 312 Specifically, a first pressure sensor trend graph P1G, a second pressure sensor P2G, and a third pressure sensor trend graph P3G are illustrated. A first pressure sensor icon P1I, a second pressure sensor icon P2I, and a third pressure sensor icon P3I are arranged in parallel and adjacent to each other on the current value side of the respective trend graphs P1G and the like.

Further, on the current value side of the trend graph P1G and the like, the graph scale M of the trend graph is also displayed in parallel and in close proximity. By arranging the first pressure sensor icon P1I and the graph scale M in this manner, the trend graph is easily visually recognized by the user.

An example of a lower-side lower display 422 of third screen 400 is illustrated in FIG. 5 with reference to FIGS. 1-4, showing a screen example of the lower-side lower display 422 of the third screen 400. For example, in this configuration, the user can change (transition) the screen to the third screen 400 in FIG. 5 by operating the screen change icon C2 from the second screen 300 in FIG. 4.

A trend graph based on the value of oxygen saturation of the oxygen saturation sensor 21, the value of temperature of the temperature sensor 23, and the value of the hematocrit sensor 22 is displayed on the lower-side lower display 422 of FIG. 5. Specifically, oxygen saturation sensor trend graphs SO2-1G and SO2-2G, a temperature trend graph TG and a hematocrit trend graph HCTG are illustrated.

On the current value side of each trend graph SO2-1 and the like, oxygen saturation sensor icons (SO2-1I) and (SO2-2I), a temperature sensor icon (TI), and a hematocrit icon (HCTI) are displayed in parallel and close to each other.

Further, on the current value side of the trend graph SO2-1G and the like, the graph scale M of the trend graph SO2-1 and the like are also displayed in parallel and adjacent to each other.

By arranging the oxygen saturation sensor icons SO2-1I and SO2-2I and the like, and the graph scale M in this manner, trend graphs that can be easily visually recognized by the user are obtained.

The present embodiment has a configuration in which the upper display 201 displays the current values of the most important parameters, the upper-side lower display 311 of the lower display 310 displays the trend graph LG and the like of the parameters displayed on the upper display 201, and the lower-side lower displays 312 and 422 are changeable and display parameters to be checked between two screens as necessary, so as to be easily observed by the user.

Further, since all the trend graphs LG and the like displayed on the lower display 320 are the trend graphs LG and the like in the same time period TS, it is possible to easily grasp whether or not the extracorporeal circulation device 1 is normally operating.

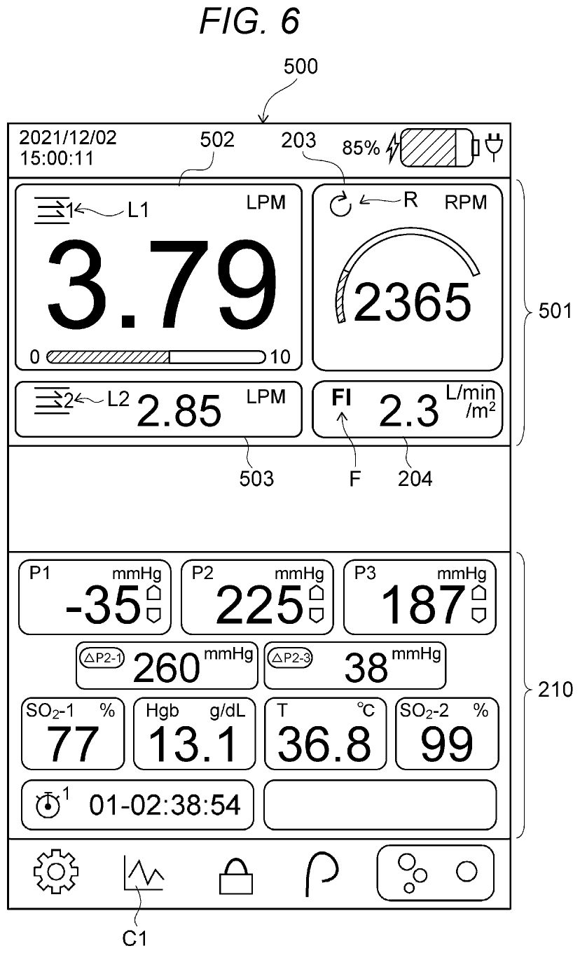

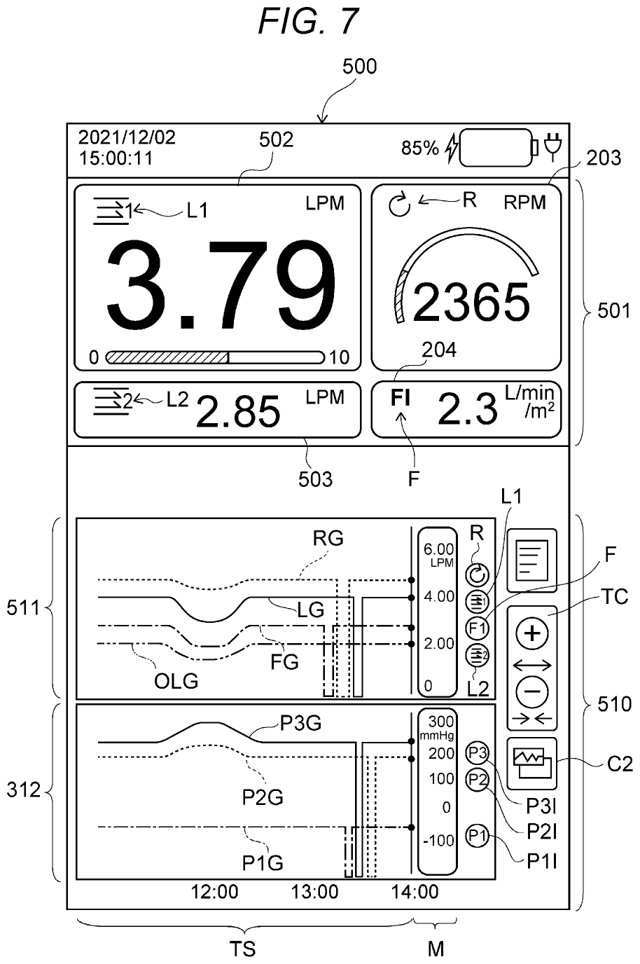

Examples when an optional flow rate sensor (not illustrated) are used are illustrated in FIGS. 6 and 7, with reference to FIGS. 1-3, showing schematic explanatory diagrams of a fourth screen 500 that displays information of an optional flow rate sensor (not shown) on the display 113. That is, unlike FIG. 3, in FIG. 6, an optional flow rate frame 503 for displaying the flow rate value (for example, 2.85 or the like) of an optional flow rate sensor (not shown) is displayed below a flow rate frame 502 for displaying the flow rate value of the flow rate sensor 7 on an upper display 501. The optional flow rate sensor (not shown) is a flow rate sensor optionally installed in the extracorporeal circulation device 1 of FIG. 1 separately from the flow rate sensor 7.

Therefore, when the user of the extracorporeal circulation device 1 sets the optional flow rate sensor to be used (enabled), the unused first screen 200 of the optional flow rate sensor in FIG. 3 is switched to the fourth screen 500 in FIG. 6, and the optional flow rate frame 503 is displayed. At this time, the optional flow rate frame 503 is displayed smaller than the flow rate frame 502 for displaying the flow rate value of the flow rate sensor 7. Therefore, in this configuration, it is possible to prevent the user from having difficulty in visually recognizing the flow rate value of the flow rate sensor 7 due to the presence of the optional flow rate frame 503.

Then, in the optional flow rate frame 503, characters of LPM indicating the flow rate of the optional flow rate sensor and an optional flow rate icon L2 are displayed together with numerical information (for example 2.85 and the like).

When the screen change icon C1 is selected while the fourth screen 500 in FIG. 6 is displayed, the screen transitions to the screen in FIG. 7. In FIG. 7, transition is made from the lower display 210 in FIG. 6 to the lower display 510 in FIG. 7. The trend graph information such as the flow rate value is displayed on the lower display 510 of FIG. 7.

Specifically, not only the flow rate trend graph LG, the rotation speed trend graph RG of the centrifugal pump 3, and the FI value trend graph FG but also an optional flow rate trend graph OLG which is a trend graph of the optional flow rate sensor, is displayed on the upper-side lower display 511 unlike the first screen 200 of FIG. 3.

On the current value side of the optional flow rate trend graph OLG, the optional flow rate icon L2 is displayed in proximity. Therefore, the user using the optional flow rate sensor 17 can easily grasp not only the numerical information but also the information of the trend graph OLG with a simple operation.

As described above, the present embodiment has a configuration in which the extracorporeal circulation information (flow rate or the like) is displayed on the display 113 so that the user can grasp or can easily grasp the extracorporeal circulation information, and the screen can be changed (transitioned) to the screen of FIG. 3, FIG. 4, FIG. 5, or the like as necessary by selecting the screen change icon C1 or the like illustrated in FIG. 3. Therefore, the user can quickly and reliably grasp necessary information even with the small display 113.

In particular, when a low-skilled person operates the extracorporeal circulation device 1 requiring skill, there is a case where a numerical value or the like to be visually recognized first cannot be easily recognized. On the other hand, this demand is particularly high at a site where the extracorporeal circulation device 1 is used with high urgency.

In this regard, in the present embodiment, even a low-skilled person can easily and quickly recognize a numerical value or the like to be visually recognized first by appropriately changing the screen of the display 113.

As described above, according to the present embodiment, since the plurality of types of non-fixed information (numerical screen, trend graph, and the like) is switched and sequentially displayed on the lower display 210 or the like, the user can easily and quickly grasp all of the plurality of pieces of non-fixed information with a size that is easy to visually recognize.

In the examples of the present embodiment, as described above, the screen of FIGS. 3 to 7 can be changed by a user's operation or the like, but the present invention further has the following configuration. That is, in a case where the time during which the user does not perform the operation has elapsed for a certain period of time, the screens of FIGS. 3, 4, and 5 are automatically changed and displayed in a predetermined order.

That is, in the above examples, the configuration in which the lower displays 210, 310, 422, and the like are changed by selecting the screen change icon C1 has been described, but the present invention is not limited thereto, and the contents of the lower display 210 are automatically changed and displayed in a predetermined order after a predetermined time elapses.

For example, when the user's operation ends and the screen operation is not performed for a certain period of time, the above-described lower displays 210, 310, 422, and the like are automatically switched and displayed. Thus, the user can grasp all of the screens of FIGS. 3 to 5 without performing an operation or the like by himself/herself.

Further, in this configuration, since there is a demand for grasping numerical values (the lower display 210) and grasping the trend graph LG and the like (the lower displays 310, 422, and the like) by the user, the time of numerical value display and the time of display of the trend graph LG and the like can be arbitrarily set.

For example, a numerical value (0 seconds (s), 3 s to 10 s), the trend graph LG, or the like (0 s, 3 s to 10 s) can be set in units of one second. However, it is not possible to set 0 seconds in both cases.

As described above, when the user intentionally checks the screen, three seconds that can be recognized are set as the shortest time, and since it is necessary to view both screens for the purpose of switching display, the maximum display time may be set to 10 seconds. Further, the user can perform setting within this range.

Since it is difficult to perform the operation if the screen is automatically changed while the user performs the operation, in this configuration, the function of automatically switching the display starts to operate in a case where the user leaves the screen for a certain period of time (for example, one minute) after the user stops performing the operation.

In addition, in this configuration, when an operation is started by touching the screen, the display switching function is stopped.

With these functions, in a case where the user does not want to perform an unnecessary operation at the time of checking at the time of periodic patient rounds where the user cannot take his/her hand off, it is possible to check the current state by numerical values and the tendency from the past to the present by a graph line without requiring an operation of the user. There is an advantage that both pieces of information can be grasped without manipulation of the screen by the user.

In the present examples, the display time of the trend graph LG and the like is three hours (11:00 to 14:00) on the second screen 300 of the display 113 of FIG. 4 and the third screen 400 of FIG. 5.

However, the present embodiment has a configuration in which the user can freely change a time axis for the display time of the trend graph LG or the like.

Specifically, the time axis of the screen can be changed by selecting the time axis icon TC of time axis + and time axis − displayed on the right side of the lower display 310 in FIG. 4.

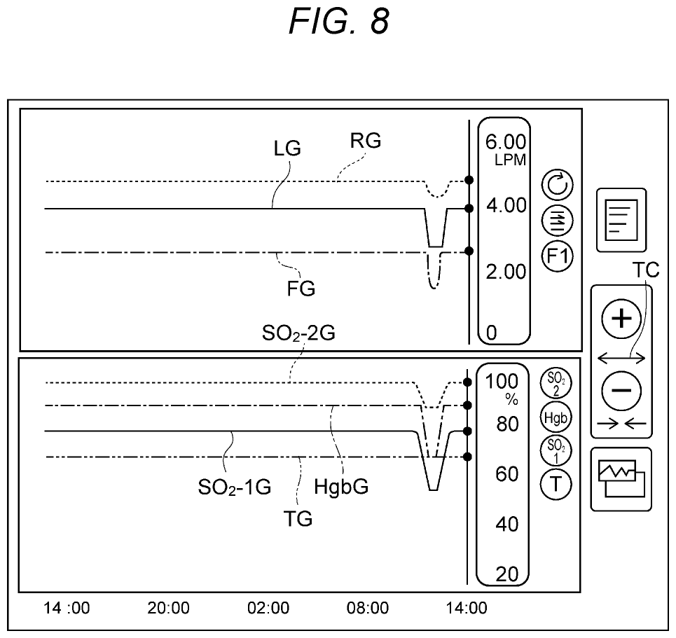

For example, when the time axis icon TC of time axis + (enlarged time axis) is selected in a state where the time axis in FIG. 4 is three hours, the screen for setting time axis to 24 hours is displayed in a changed manner as illustrated in FIG. 8.

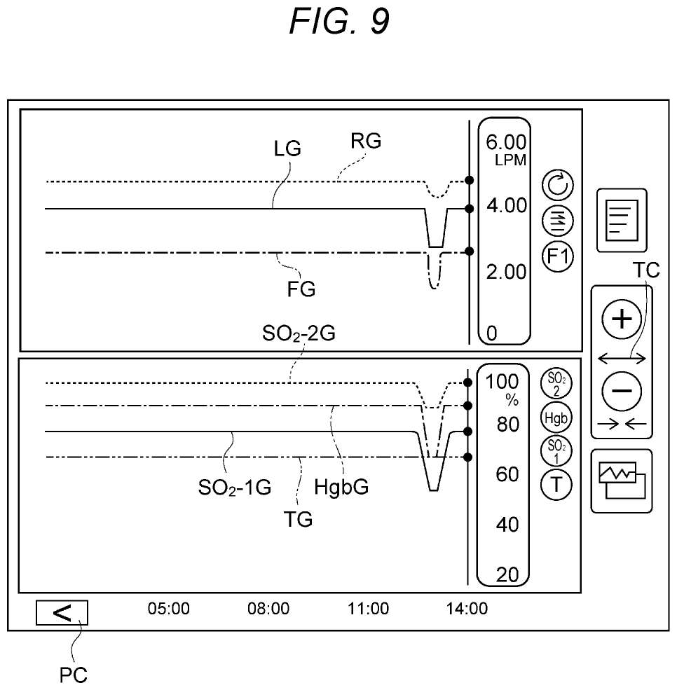

Further, when time axis − (time axis reduction) is selected in the state of the time axis of 24 hours in FIG. 8, the screen is switched to a screen in which the time axis is 12 hours as illustrated in FIG. 9.

Further, when it is desired to display a past portion that is not displayed on the screen in a state where the time axis of FIG. 8 is 12 hours, it is possible to display data of a desired time period, for example, the time axis of 14:00 to 02:00 on the screen by selecting a past time axis icon PC of FIG. 9.

As described above, according to the present embodiment, since the user can display the trend graph information on a desired time axis each time, the configuration is easy to use.

In the examples of the present embodiment, since it is possible to Perform the setting change of the numerical value set for each piece of extracorporeal circulation information displayed on the display 113 (upper limit value of alarm or the like) by a simple method, the following description will be made.

In particular, in the present embodiment, non-fixed information (extracorporeal circulation information other than the flow rate value, the rotation speed of centrifugal pump 3, and the FI value), for example, setting change of an alarm such as a pressure value, oxygen saturation, and hematocrit value will be described as an example.

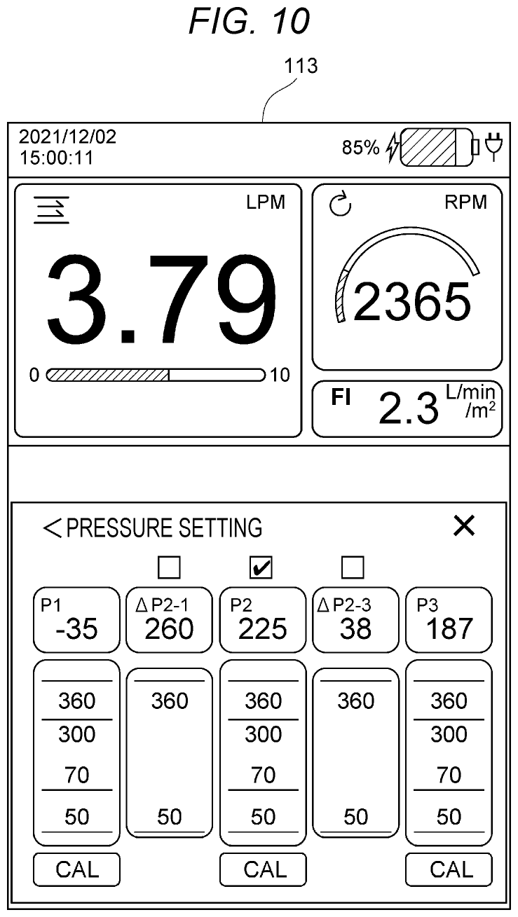

An example of a pressure alarm setting will be discussed. When the user selects inside of a frame 211 in a display portion of the pressure sensor such as P1 of the lower display 210 of FIG. 3, the screen transitions to a pressure setting screen as illustrated in FIG. 10.

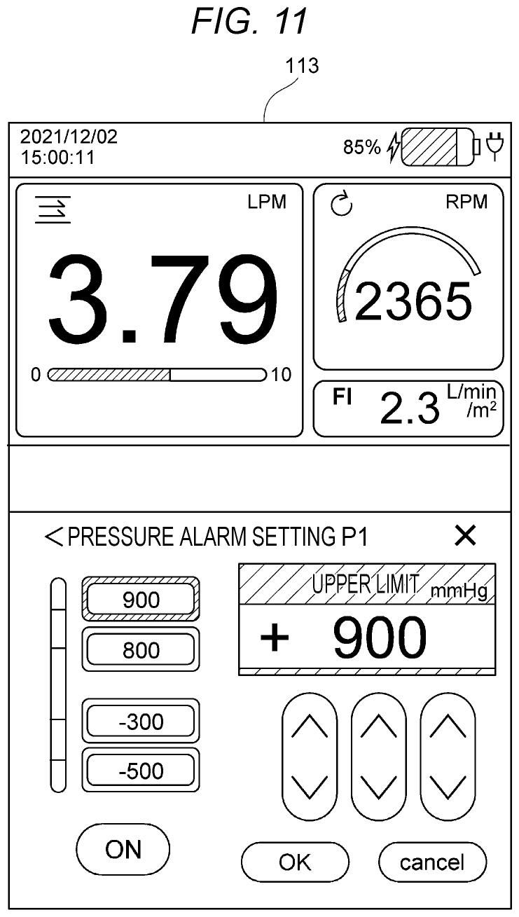

Then, when the user selects the numerical value part on the pressure setting screen of FIG. 10, a pressure alarm setting screen is opened as illustrated in FIG. 11.

On this pressure setting screen, for example, an upper limit set value (900 mmHg) can be changed.

As described above, in the present embodiment, it is possible to easily and quickly change and register the pressure value of the pressure alarm setting by only selecting the corresponding portion of the screen.

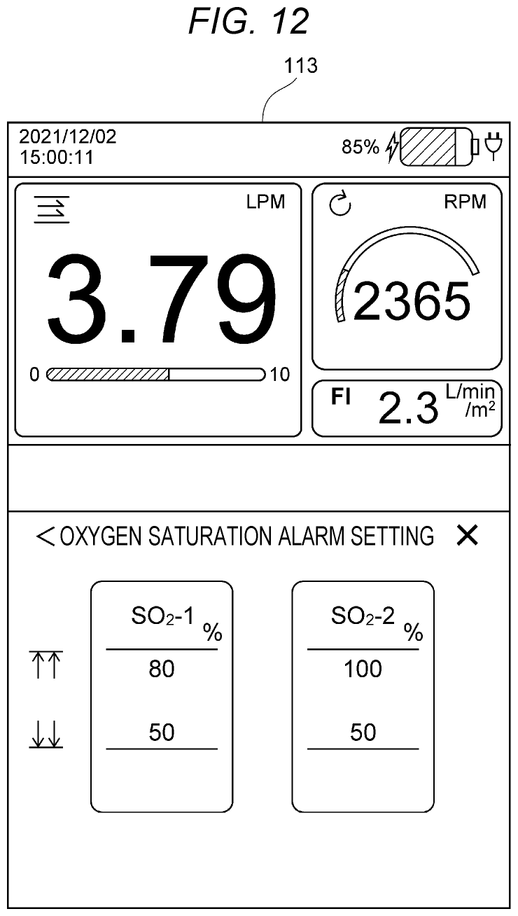

An example of an oxygen saturation alarm setting will be discussed. When the user selects a frame 212 of the oxygen saturation such as SO2-1 of the lower display 210 in FIG. 3, the screen transitions to an oxygen saturation alarm setting screen as illustrated in FIG. 12.

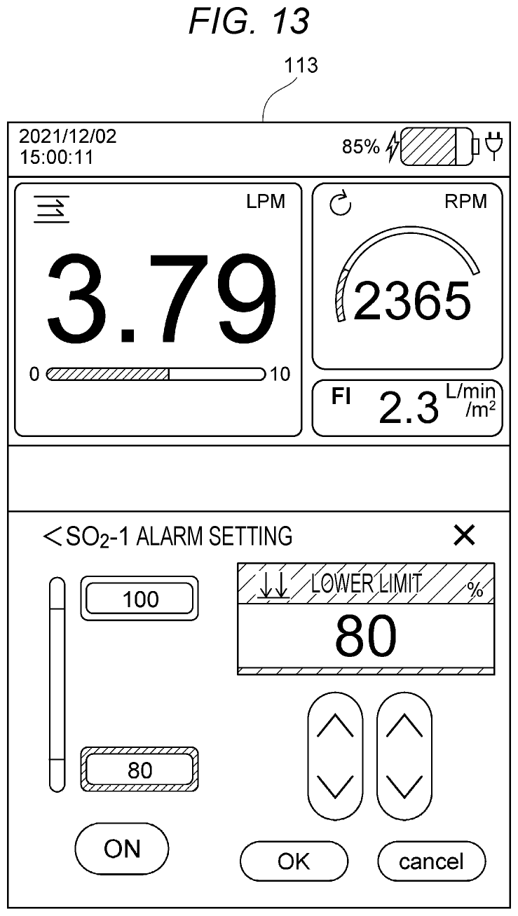

Then, when the user selects the numerical value part of SO2-1 on the oxygen saturation alarm setting screen, the screen transitions to a screen of SO2-1 alarm setting as illustrated in FIG. 13.

On the SO2-1 alarm setting screen, for example, a lower limit set value (80%) can be changed. As described above, in the present embodiment, it is possible to easily and quickly change and register the setting of the oxygen saturation by only selecting the corresponding portion of the screen.

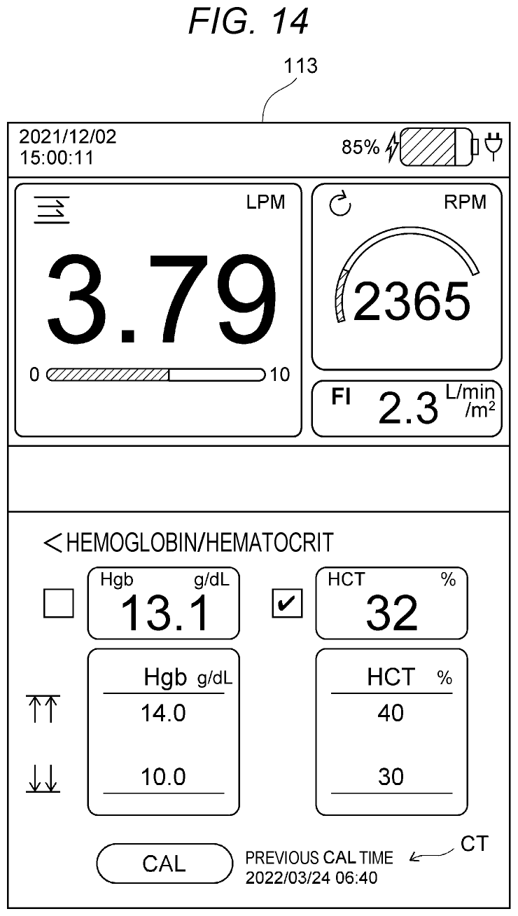

An example of a hemoglobin/hematocrit setting will be discussed. When the user selects a frame 213 of Hgb in the lower display 210 in FIG. 3, the screen transitions to a hemoglobin/hematocrit value setting screen as illustrated in FIG. 14.

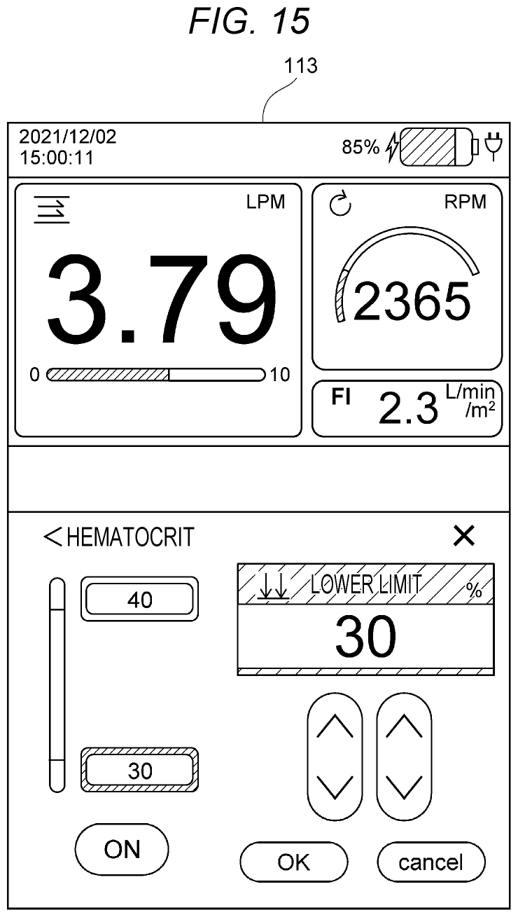

The user selects Hgb or HCT in FIG. 14. For example, when HCT is selected, the screen transitions to a value setting screen for the lower limit value or upper limit value of hematocrit as illustrated in FIG. 15.

On the SO2-1 alarm setting screen, for example, a lower limit set value (30%) can be changed. As described above, in the present embodiment, the hematocrit value can be easily and quickly changed and registered by only selecting the corresponding portion of the screen.

An example of a calibration time display switching will be discussed. In hemoglobin/hematocrit illustrated in FIG. 14, calibration CAL is required, and thus, as illustrated in FIG. 14, for example, calibration time 2022 Mar. 24 06:40 (CT) which is the previous calibration execution time is displayed as the time display area.

However, such time display has a problem that the user cannot instantaneously grasp the elapsed time from the previous CAL even when the user visually recognizes the time display. Accordingly, the present embodiment has a configuration in which it is possible to perform display such as 10 hours before the previous calibration time and display elapsed time information starting from the current time.

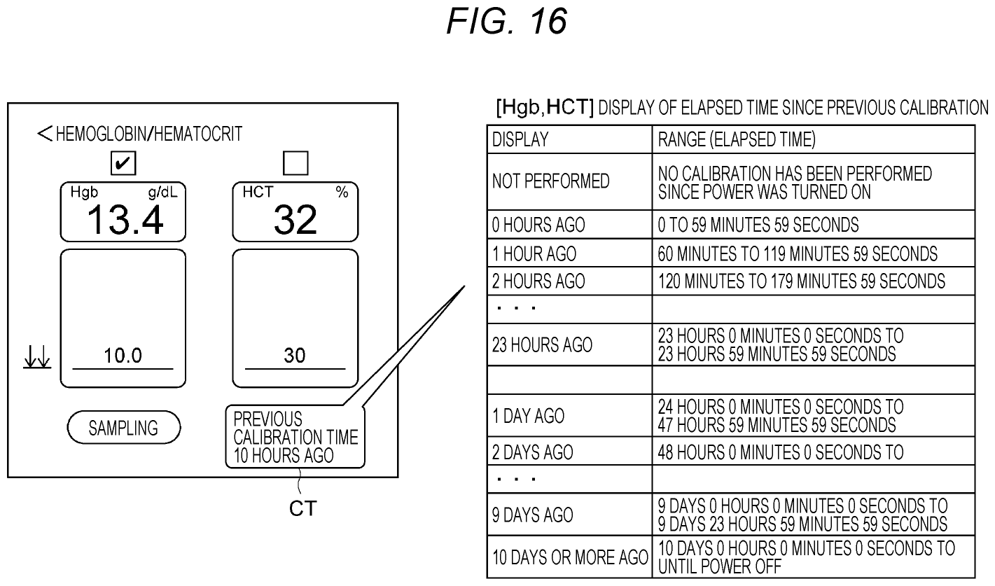

FIG. 16 is a schematic explanatory diagram illustrating an example of a mode of elapsed time display of CAL implementation. The management device 10 stores data for display of elapsed time from previous calibration. Then, when the user displays the setting change screen illustrated in FIG. 14 in order to change the setting of the hemoglobin/hematocrit value, the screen example illustrated on the left side of FIG. 16 is obtained instead of FIG. 14.

That is, the time from the current time to the previous CAL execution is displayed instead of the calibration execution time. Specifically, the determination is made based on which item in the table of FIG. 16 the execution time of the previous CAL and the time until the present (setting time) corresponds to. For example, in a case where the period from the previous CAL to the present is 90 minutes, 1 hour ago is displayed.

As described above, according to the present embodiment, it is possible to instantly grasp the rough elapsed time from the previous CAL on the hemoglobin/hematocrit setting change screen.

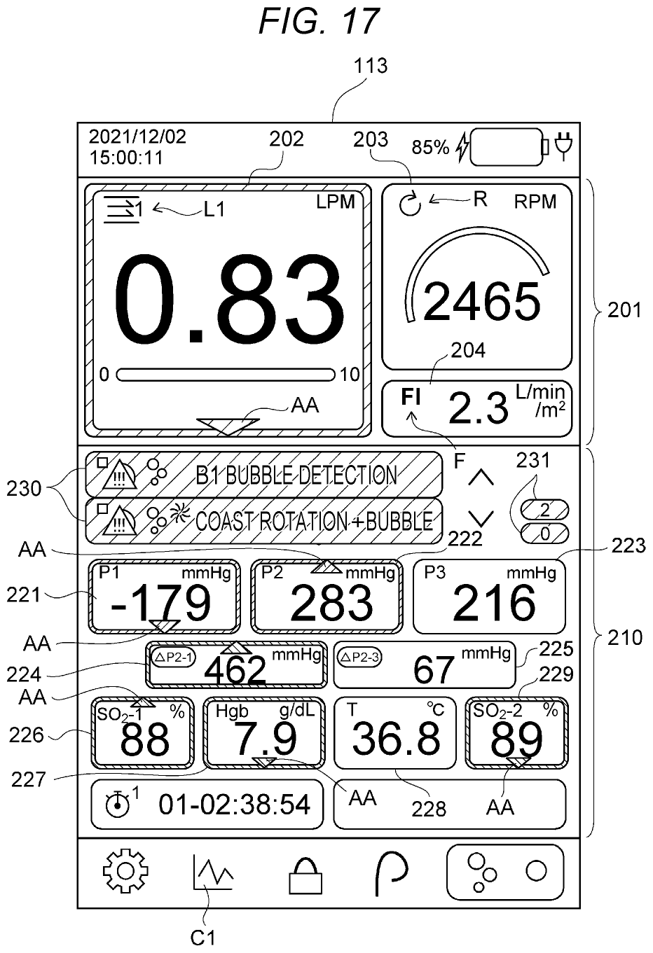

An example of an alarm display will now be discussed. FIG. 17 is a schematic explanatory diagram illustrating an example of the display 113 in a state where an alarm is displayed.

In the example of the present embodiment, there is provided an abnormal value information detection unit 120 (FIG. 2) that detects abnormal value information regarding the flow rate value, the rotation speed of centrifugal pump 3, and the FI value of the upper display 201, and the first pressure sensor value P1, the second pressure sensor value P2, the third pressure sensor value P3, oxygen saturation sensor values SO2-1 and SO2-2, hemoglobin value Hgb, temperature value T, and the like displayed on the lower display 210, which are illustrated in FIG. 17.

Further, the flow rate value and the like of the upper display 201 described above are displayed while being surrounded by the frame parts 202, 203, and 204, respectively.

In addition, the first pressure sensor value P1, the second pressure sensor value P2, the third pressure sensor value P3, the oxygen saturation sensor value SO2-1, the oxygen saturation sensor SO2-2, the hemoglobin value Hgb, and the temperature value T of the lower display 210 are also displayed by being respectively surrounded by frame parts 221 to 229 as illustrated in FIG. 17.

Then, when the abnormal value information detection unit 120 in FIG. 2 detects an abnormality, the frame for which the abnormality has occurred is set as an abnormal frame part, and is indicated on the display 113 by an alarm color which is a color different from the background color of the display 113.

This alarm color is indicated in different colors depending on the priority of the alarm, and in FIG. 17, in a case where the level of the alarm is high, it is indicated in red (right-inclined hatching), and then in a case where the level is relatively lower (than high), it is indicated in yellow (left-inclined hatching).

In the example of FIG. 17, the frame part 202 of the flow rate value of the upper display 201 and the frame parts 221 and 222 of the first pressure sensor value P1 and the second pressure sensor value P2 of the lower display 210 are displayed in red (right-inclined hatching) alarm colors.

Further, frame parts 226, 227, and 229, and the like such as the oxygen saturation sensor value SO2-1, the oxygen saturation sensor SO2-2, and the hemoglobin value Hgb of the lower display 210 are displayed in yellow (left-inclined hatching) alarm colors.

In this way, by indicating red, yellow, or the like different from the background color, the user can instantaneously grasp the presence or absence and the degree of the alarm.

Further, it is also possible to guide a high priority portion to be clicked (confirmed).

As illustrated in FIG. 17, red or yellow indicates abnormality, and an icon part indicating the abnormality content, for example, alarm content icon AA is also displayed.

Specifically, an upward arrow is displayed when the abnormal value exceeds the upper limit value, and a downward arrow is displayed when the abnormal value exceeds the lower limit value.

In the example of FIG. 17, an upward arrow is displayed in the frame part 224, the frame part 222 of the second pressure sensor value P2, the frame part 226 of the oxygen saturation sensor value SO2-1, and the like.

Further, alarm content icons AA indicated by downward arrows are displayed in the frame parts 202, 221, 227, 229, and the like of the flow rate frame, the first pressure sensor value P1, hemoglobin value Hgb, and oxygen saturation SO2-2, respectively.

Therefore, by visually recognizing the alarm content icon and the direction of the arrow, the user can instantaneously grasp the occurrence and content of the abnormality.

Furthermore, in the present embodiment, as illustrated in FIG. 17, for example, abnormality content text information 230 (for example, B1 bubble detection alarm, coast rotation+bubble alarm, and the like) which is an abnormality information display part (abnormality information content) is scrolled and displayed.

At the same time, the number of occurrences of abnormal values (number information) 231 (for example, red 2, yellow 0, and the like in FIG. 17) is also displayed.

At this time, the number of occurrences of abnormal values 231 is displayed on the display 113 including other hidden abnormal values for which no alarm is actually displayed in red or yellow.

The user can more specifically grasp the abnormality content and can accurately grasp the number of abnormalities.

Further, the number of pieces of abnormal value information can also be accurately grasped for each degree.

Hereinafter, a display method of the abnormality content text information 230 according to the present embodiment will be specifically described.

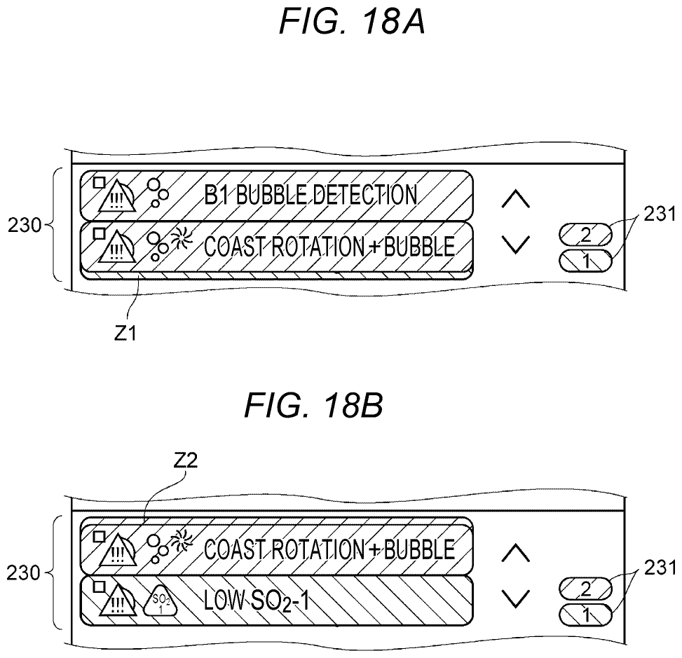

FIGS. 18A and 18B are schematic explanatory diagrams illustrating examples of displaying three pieces of abnormality content text information on the display 113. FIG. 18A is a schematic explanatory diagram illustrating an example of displaying B1 bubble detection (red alarm color) and coast rotation+bubble (red alarm color) corresponding to two of the three pieces. FIG. 18B is a schematic explanatory diagram illustrating an example of displaying the coast rotation+bubble (red alarm color) and low SO2-1 (yellow alarm color) corresponding to two out of the three pieces.

The present embodiment has a configuration in which up to two pieces of the abnormality content text information 230 can be displayed, and thus they are displayed on the display 113 as follows.

For example, the plurality of pieces of the abnormality content text information 230 is configured to be displayed in order from the top to the bottom in descending order of priority in which the alarm has been generated.

In FIG. 18A, as illustrated, the B1 bubble detection (red alarm color) which is an alarm with a high priority to be preferentially resolved is displayed in the upper part, and the coast rotation+bubble (red alarm color) which is an alarm with the next highest priority is displayed in the lower part.

At this time, the low SO2-1 (yellow alarm color) which is the third highest priority alarm cannot be simultaneously displayed. Thus, in the present embodiment, as illustrated in FIG. 18A, a band-shaped zone Z1 is arranged below the coast rotation+bubble (red alarm color) displayed at the second lower part. In this configuration, in the zone Z1, the low SO2-1 (yellow alarm color) cannot be displayed, but only the alarm color (yellow) is displayed without describing characters or the like. Therefore, the user can grasp that, in the order of priority, an alarm having the third highest priority has also occurred, and further, the user can instantaneously grasp the degree (red or yellow) of the alarm.

At the same time, as in FIG. 17, the number of occurrences of abnormal values 231 is displayed by color (by degree of alarm), so that the user can instantaneously grasp the degree of alarm and the number of alarms.

Further, the user can scroll the display of FIG. 18A.

FIG. 18B is a schematic explanatory diagram illustrating a state in which the display of the abnormality content text information 230 in FIG. 18A is scrolled and displayed.

In FIG. 18B, the coast rotation+bubble (red alarm color) which is the lower part in 18A is displayed in the upper part, and the low SO2-1 (yellow alarm color) displayed in the zone Z1 is displayed in 18A in the lower part.

In this case, in 18A, the B1 bubble detection (red alarm color) displayed in the upper part cannot be displayed, and thus, as illustrated in 18B, in this configuration, a band-shaped zone Z2 is arranged above the coast rotation+bubble (red alarm color) in the upper part, and only the alarm color (red) is displayed without displaying characters or the like.

Therefore, the user can grasp that the first alarm is generated in the priority order, and further, the user can instantaneously grasp the degree (red or yellow) of the alarm.

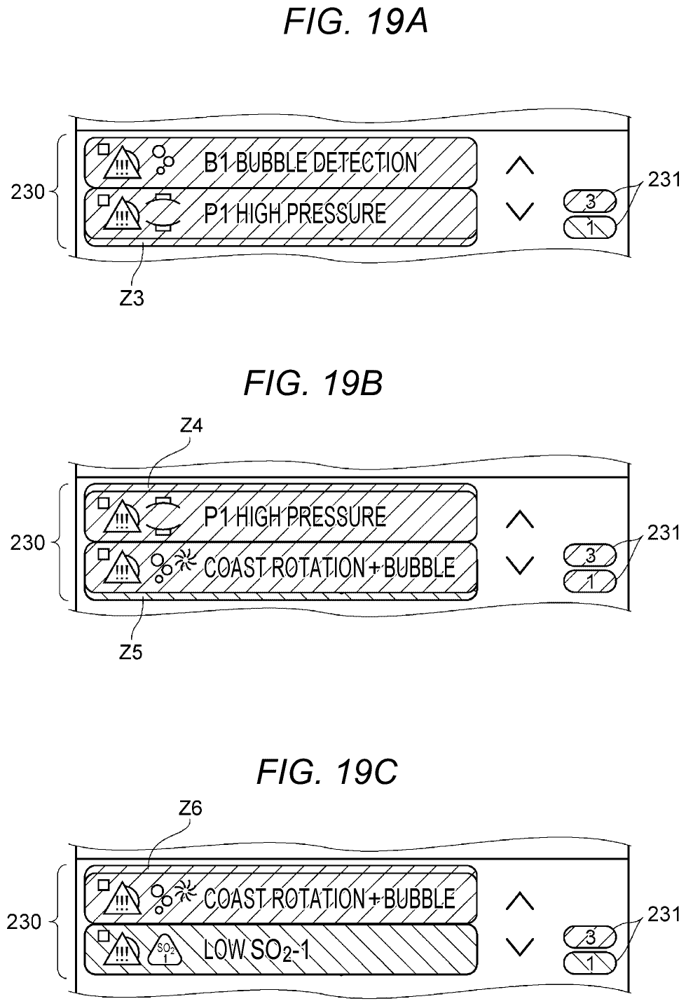

FIGS. 19A, 19B and 19c are schematic explanatory diagrams illustrating examples in which four pieces of abnormality content text information are displayed on the display 113, and 19A to 19C are schematic explanatory diagrams illustrating the scroll display.

In the examples of FIGS. 19A-19C, four alarms are generated, and specifically, the following alarms are generated in order of occurrence time.

That is, the B1 bubble detection (red alarm color), the coast rotation+bubble (red alarm color), the low SO2-1 (yellow alarm color), and P1 high pressure (red alarm color) are generated in this order.

On the other hand, in a case where the number of pieces of the abnormality content text information 230 that can be displayed on the display 113 is limited to two, in the present embodiment, conversion and display are performed not in the alarm generation order, but in the descending order of priority of alarms, that is, in the order of the B1 bubble detection (red alarm color), the P1 high pressure (red alarm color), the coast rotation+bubble (red alarm color), and the low SO2-1 (yellow alarm color).

Specifically, since scroll display is performed as follows, the following description will be made with reference to FIG. 19A to 19C.

First, in FIG. 19A, the B1 bubble detection (red alarm color) which is the alarm with the highest priority is displayed in the upper part, and the P1 high pressure (red alarm color) which is the alarm with the second highest priority is displayed in the lower part.

Then, the third highest priority alarm coast rotation+bubble (red alarm color) is indicated by a band-shaped zone Z3 displaying only the alarm color (red), and the fourth highest priority low SO2-1 (yellow alarm color) is not displayed.

However, in this configuration, since the number of occurrences of abnormal values 231 in FIG. 19A indicates that three red cases and one yellow case have occurred, the user can grasp that four alarms have occurred in total and the degree of the alarm (three cases are red and one case is yellow).

Next, when the user performs scroll display, FIG. 19B is displayed. In FIG. 19B, the P1 high pressure (red alarm color) is displayed in the upper part, and the coast rotation+bubble (red alarm) is displayed in the lower part. Then, the B1 bubble detection (red alarm color) is displayed on the upper part of the P1 high pressure (red alarm color) as a band-shaped zone Z4 of only the alarm color.

On the other hand, the low SO2-1 (yellow alarm color) is displayed as a band-shaped zone Z5 of only the alarm color under coast rotation+bubble (red alarm color) in the lower part.

Next, when the user performs scroll display, FIG. 19C is displayed.

In FIG. 19C, coast rotation+bubble (red alarm color) is displayed in the upper part, and low SO2-1 (yellow alarm color) is displayed in the lower part. Then, the P1 high pressure (red alarm color) is displayed as a band-shaped zone Z6 of only the alarm color on the upper part of the coast rotation+bubble (red alarm).

As described above, according to the present embodiment, by performing scroll display of the abnormality content text information 230 displayed on the display 113, the user can easily confirm all the alarms.

Further, in the present embodiment, although only one belt-shaped zone Z1 or the like is displayed on the upper side or the lower side of an alarm accompanied by display of characters or the like, the present invention is not limited thereto, and a plurality of belt-shaped zones Z1 or the like may be arranged.

In the embodiment described above, the case of being implemented as a device has been described as an example, but the present invention is not limited thereto, and as a non-transitory computer readable program instructions that can be executed by a computer, the program may be stored and distributed in a storage medium such as a magnetic disk (removable disk, hard disk, and the like), an optical disk (CD-ROM, DVD, or the like), a magneto-optical disk (MO), or a semiconductor memory.

The storage medium may be any storage medium that can store a program and is readable by a computer. A storage format of the storage medium is not particularly limited.

In addition, an operating system (OS) running on a computer, database management software, middleware (MW) such as network software, or the like on the basis of an instruction of a program installed in the computer from a storage medium may execute a part of each process for implementing the present embodiment.

Furthermore, the storage medium in the present invention is not limited to a medium independent of a computer and includes a storage medium in which a program transmitted through a LAN, the Internet, or the like is downloaded and stored or temporarily stored.

In addition, the computer in the present invention only needs to execute each processing in the present embodiment on the basis of a program stored in a storage medium, and may be a device including one personal computer or the like, or may be a system in which a plurality of devices is connected to a network, or the like.

Further, the computer in the present invention is not limited to a personal computer, and includes an arithmetic processing device and an oxygenator such as a microcomputer included in an information processing device, and collectively refers to a device and an apparatus capable of implementing the functions of the present invention by a program.

The embodiment of the present invention has been described above. However, the present invention is not limited to the embodiments, and various modifications can be made without departing from the scope of the claims. The configurations of the embodiments may be partially omitted or optionally combined to make different configurations from the aforementioned configurations.

### REFERENCE SIGNS LIST

1 Extracorporeal circulation system
2 Oxygenator
3 Centrifugal pump
4 Drive motor
Venous side catheter
6 Arterial side catheter
7 Flow rate sensor
8, 9 Connector
10 Controller
11 Blood removal tube
12 Blood feeding tube
21 Oxygen saturation sensor
22 Hematocrit sensor
23 Temperature sensor
24 Hemoglobin amount measuring instrument
111 Control unit
112 Communication device
113 Display
114 Various information input device
115 Clock
116 Trend graph generation unit
117 Extracorporeal circulation information storage unit
118 Various information storage unit
119 FI information generation unit
120 Abnormal value information detection unit
114 Various information input device
115 Clock
116 Trend graph generation unit
117 Extracorporeal circulation information storage unit
118 Various information storage unit
119 FI information generation unit
120 Abnormal value information detection unit
200 First screen
201 Upper display
202 Flow rate frame
203 Centrifugal pump frame
204 FI frame
210, 310 Lower display
211 Frame of display portion of pressure sensor P1
212 Oxygen saturation frame
213 Frame of “Hgb”
221, 222, 223, 224, 225, 226, 227, 228, 229 Frame part
230 Abnormality content text information
231 Number of occurrences of abnormal values
300 Second screen
310 Lower display
311 Upper-side lower display
312, 422 Lower-side lower display
400 Third screen
IR Circulation circuit
P Patient
P1 First pressure sensor
P2 Second pressure sensor
P3 Third pressure sensor
AA Alarm content icon
C1, C2 Screen change icon
TC Time axis icon
PC Past time axis icon

What is claimed is:

1. An extracorporeal circulation management device that manages an extracorporeal circulation device, which includes an oxygenation device, a pump unit, and an extracorporeal circulation circuit, the extracorporeal circulation management device comprising:
a display unit that displays various types of information regarding the extracorporeal circulation device, the display unit comprising:
a first display area in which fixed information, which is information fixed in advance, is displayed; and
a second display area, configured such that displayed information is changeable by a user, and trend graph information generated over a time period from the fixed information displayed in the first display area is configured to be displayed in graphical form in the second display area, and non-fixed information related to the fixed information is configured to be displayed in the second display area.

2. The extracorporeal circulation management device according to claim 1, wherein a setting screen configured to change the types and time periods of the non-fixed information displayed in the second display area is displayable instead of the non-fixed information.

3. The extracorporeal circulation management device according to claim 2, wherein
the setting screen includes a time display area, and
in the time display area, elapsed time information starting from a current setting time is displayable.

4. The extracorporeal circulation management device according to claim 1, wherein a time axis of time information for displaying the trend graph information is changeable.

5. The extracorporeal circulation management device according to claim 1, further comprising an abnormal value information detection unit configured to detect abnormal value information regarding the fixed information and the non-fixed information, wherein the fixed information and the non-fixed information are displayed by being divided by different frame parts for each of the fixed information and each of the non-fixed information, and when the abnormal value information detection unit detects an abnormality in at least one of the fixed information or in at least one of the non-fixed information, the abnormal value information detection unit displays an abnormal frame part around the abnormality that is detected in an alarm color different from a background color of the display unit.

6. The extracorporeal circulation management device according to claim 5, wherein the alarm color is displayed in a plurality of different colors, with each color of the plurality of colors varying based on a degree of priority of the alarm.

7. The extracorporeal circulation management device according to claim 5, wherein an icon part indicating abnormality content is displayed in the abnormal frame part.

8. The extracorporeal circulation management device according to claim 7, wherein the icon part is an upward arrow when the abnormality content is higher than an upper limit value, and is a downward arrow when the abnormality content is lower than a lower limit value.

9. The extracorporeal circulation management device according to claim 5, comprising:
an abnormality information display part that is configured to display abnormality information content related to the abnormal frame part, wherein,
the abnormality information display part is configured to be scrolled and displayed, and
the abnormality information display part is configured to simultaneously display information of a number of pieces of abnormal value information detected by the abnormal value information detection unit within a certain period of time.

10. The extracorporeal circulation management device according to claim 1, wherein when a plurality of pieces of pressure information is displayed as the non-fixed information in the second display area, a plurality of pieces of the pressure information is displayed in parallel in an order corresponding to a blood flowing direction of the extracorporeal circulation device.

11. The extracorporeal circulation management device according to claim 1, wherein a plurality of types of the non-fixed information is configured to be changed by the user as to which of the plurality of types of the non-fixed information is displayed on the second display area and displayed in a predetermined order.

12. An extracorporeal circulation device comprising:
an oxygenation device;
a pump unit;
an extracorporeal circulation circuit; and
a management device, wherein the management device includes a display unit that displays various types of information regarding the extracorporeal circulation device, the display unit including a first display area in which fixed information, which is information fixed in advance, is displayed,
a second display area configured such that displayed information is changeable by a user, and
trend graph information generated over a time period from the fixed information displayed in the first display area is configured to be displayed in graphical form in the second display area, and non-fixed information related to the fixed information is configured to be displayed in the second display area.

13. The extracorporeal circulation device according to claim 12, wherein a setting screen configured to change the types and time periods of the non-fixed information displayed in the second display area is displayable instead of the non-fixed information.

14. The extracorporeal circulation device according to claim 13, wherein
the setting screen includes a time display area, and
in the time display area, elapsed time information starting from a current setting time is displayable.

15. The extracorporeal circulation device according to claim 12, wherein a time axis of time information for displaying the trend graph information is changeable.

16. The extracorporeal circulation device according to claim 12, further comprising an abnormal value information detection unit configured to detect abnormal value information regarding the fixed information and the non-fixed information, wherein the fixed information and the non-fixed information are displayed by being divided by different frame parts for each of the fixed information and each of the non-fixed information, and when the abnormal value information detection unit detects an abnormality in at least one of the fixed information or in at least one of the non-fixed information, the abnormal value information detection unit displays an abnormal frame part around the abnormality that is detected in an alarm color different from a background color of the display unit.

17. A method of operating an extracorporeal circulation management device that manages an extracorporeal circulation device including an oxygenation device, a pump unit, and an extracorporeal circulation circuit, the method comprising:
displaying various types of information regarding the extracorporeal circulation device on a display unit of the management device;
displaying fixed information, which is information fixed in advance, in a first display area of the display unit,
displaying user changeable display information in a second display area of the display unit, and
generating trend graph information over a time period from the fixed information displayed in the first display area, and displaying, in graphical form, the trend graph information in the second display area, and displaying types of non-fixed information related to the fixed information in the second display area.

18. The method according to claim 17, further comprising:
Displaying a plurality of pieces of pressure information as the non-fixed information in the second display area in parallel in an order corresponding to a blood flowing direction of the extracorporeal circulation device.

19. The method according to claim 17, further comprising:
Changing, by a user, a plurality of types of the non-fixed information, wherein which of the plurality of types of the non-fixed information is displayed on the second display area is displayed in a predetermined order.
